(19) United States
(12) Patent Application Publication
(10) Pub. No.: US 2016/0376148 A1
(43) Pub. Date: Dec. 29, 2016

(54) Method for Producing Hydrocarbons by Oxidative Coupling of Methane without Catalyst

(71) Applicant: Sabic Global Technologies, B.V., BERGEN OP ZOOM (NL)

(72) Inventors: Aghaddin MAMEDOV, Sugar Land, TX (US); Vidya Sagar Reddy SARSANI, Pearland, TX (US); Istvan LENGYEL, Lake Jackson, TX (US); David WEST, Bellaire, TX (US)

(21) Appl. No.: 15/180,957

(22) Filed: Jun. 13, 2016

Related U.S. Application Data
(60) Provisional application No. 62/183,456, filed on Jun. 23, 2015.

(51) Int. Cl. C01B 3/36; C01B 3/50; F25J 3/02; C07C 2/82

(52) U.S. Cl. CPC C01B 3/36; C07C 2/82; C01B 3/506; F25J 3/0223; F25J 3/0233; C01B 2203/0211; C01B 2203/046; C01B 2203/048; C01B 2203/1241

(57) ABSTRACT

A method for producing olefins and synthesis gas comprising (a) introducing a reactant mixture to a reactor, wherein the reactant mixture comprises methane (CH₄) and oxygen (O₂), wherein the reactor is characterized by a reaction temperature of from about 700° C. to about 1,100° C.; (b) allowing at least a portion of the reactant mixture to react via an oxidative coupling of CH₄ reaction to form a product mixture, wherein the product mixture comprises primary products and unreacted methane, wherein the primary products comprise C₂₊ hydrocarbons and synthesis gas, wherein the C₂₊ hydrocarbons comprise olefins, and wherein a selectivity to primary products is from about 70% to about 99%; and (c) recovering at least a portion of the product mixture from the reactor.

### CROSS-REFERENCE TO RELATED APPLICATIONS

The present application is a non-provisional of and claims priority to U.S. Provisional Patent Application No. 62/183,456 filed Jun. 23, 2015 and entitled “Method for Producing Hydrocarbons by Oxidative Coupling of Methane Without Catalyst,” which application is incorporated by reference herein in its entirety.

### TECHNICAL FIELD

The present disclosure relates to methods of producing hydrocarbons, more specifically methods of producing olefins by oxidative coupling of methane.

### BACKGROUND

Hydrocarbons, and specifically olefins such as ethylene, can be typically used to produce a wide range of products, for example, break-resistant containers and packaging materials. Currently, for industrial scale applications, ethylene is produced by heating natural gas condensates and petroleum distillates, which include ethane and higher hydrocarbons, and the produced ethylene is separated from a product mixture by using gas separation processes.

Ethylene can also be produced by oxidative coupling of the methane (OCM) as represented by Equations (I) and (II):

2CH₄+O₂→C₂H₄+2H₂O ΔH=−67 kcal/mol (I)

2CH₄+½O₂→C₂H₄+H₂O ΔH=−42 kcal/mol (II)

Oxidative conversion of methane to ethylene is exothermic. Excess heat produced from these reactions (Equations (I) and (II)) can push conversion of methane to carbon monoxide and carbon dioxide rather than the desired C₂ hydrocarbon product (e.g., ethylene):

CH₄+1.5O₂→CO+2H₂O ΔH=−124 kcal/mol (III)

CH₄+)₂→CO₂+2H₂O ΔH=−192 kcal/mol (IV)

The excess heat from the reactions in Equations (III) and (IV) further exasperate this situation, thereby substantially reducing the selectivity of ethylene production when compared with carbon monoxide and carbon dioxide production.

Additionally, while the overall OCM is exothermic, catalysts are used to overcome the endothermic nature of the C—H bond breakage. The endothermic nature of the bond breakage is due to the chemical stability of methane, which is a chemically stable molecule due to the presence of its four strong tetrahedral C—H bonds (435 kJ/mol). When catalysts are used in the OCM, the exothermic reaction can lead to a large increase in catalyst bed temperature and uncontrolled heat excursions that can lead to catalyst deactivation and a further decrease in ethylene selectivity. Furthermore, the produced ethylene is highly reactive and can form unwanted and thermodynamically favored oxidation products.

There have been attempts to control the exothermic reaction of the OCM by using alternating layers of selective OCM catalysts; through the use of fluidized bed reactors; and/or by using steam as a diluent. However, these solutions are costly and inefficient. For example, a large amount of water (steam) is required to absorb the heat of the reaction. Thus, there is an ongoing need for the development of OCM processes.

### BRIEF SUMMARY

Disclosed herein is a method for producing olefins and synthesis gas comprising (a) introducing a reactant mixture to a reactor, wherein the reactant mixture comprises methane (CH₄) and oxygen (O₂), wherein the reactor is characterized by a reaction temperature of from about 700° C. to about 1,100° C., (b) allowing at least a portion of the reactant mixture to react via an oxidative coupling of CH₄ reaction to form a product mixture, wherein the product mixture comprises primary products and unreacted methane, wherein the primary products comprise C₂₊ hydrocarbons and synthesis gas, wherein the C₂₊ hydrocarbons comprise olefins, and wherein a selectivity to primary products is from about 70% to about 99%, and (c) recovering at least a portion of the product mixture from the reactor.

Also disclosed herein is a method for producing ethylene and synthesis gas comprising (a) introducing a reactant mixture to a reactor, wherein the reactant mixture comprises methane (CH₄), oxygen (0₂) and hydrogen (H₂), wherein the reactant mixture is characterized by a CH₄/O₂ molar ratio of from about 14:1 to about 18:1, wherein the reactant mixture is characterized by a CH₄/H₂ molar ratio of from about 8:1 to about 15:1, wherein the reactant mixture is characterized by a O₂/H₂ molar ratio of from about 5:1 to about 8:1, wherein the reactor is characterized by a reaction temperature of from about 950° C. to about 1,000° C., wherein the reactor is characterized by a residence time of from about 250 milliseconds to about 750 milliseconds, and wherein the reactor excludes a catalyst, (b) allowing at least a portion of the reactant mixture to react via an oxidative coupling of CH₄ reaction to form a product mixture, wherein the product mixture comprises primary products and unreacted methane, wherein the primary products comprise C₂₊ hydrocarbons and synthesis gas, wherein the C₂₊ hydrocarbons comprise ethylene, and wherein a selectivity to primary products is from about 90% to about 99%, (c) recovering at least a portion of the product mixture from the reactor, and (d) separating at least a portion of the ethylene from the product mixture by cryogenic distillation to yield recovered ethylene.

Further disclosed herein is a method for producing olefins and synthesis gas comprising (a) introducing a reactant mixture to a reactor, wherein the reactant mixture comprises methane (CH₄), oxygen (O₂), and hydrogen (H₂) used as an initiator of gas phase reactions, wherein the reactor is characterized by a reaction temperature of from about 700° C. to about 1,100° C., (b) allowing at least a portion of the reactant mixture to react via an oxidative coupling of CH₄ reaction to form a product mixture, wherein the product mixture comprises primary products and unreacted methane, wherein the primary products comprise C₂₊ hydrocarbons and synthesis gas, wherein the C₂₊ hydrocarbons comprise olefins, and wherein a selectivity to primary products is from about 70% to about 99%, and (c) recovering at least a portion of the product mixture from the reactor.

### BRIEF DESCRIPTION OF THE DRAWINGS

For a detailed description of the preferred embodiments of the disclosed methods, reference will now be made to the accompanying drawing in which:

FIG. 1 displays a schematic of a multi-stage reactor for oxidative coupling of methane.

### DETAILED DESCRIPTION

Disclosed herein are methods for producing olefins and synthesis gas comprising (a) introducing a reactant mixture to a reactor, wherein the reactant mixture comprises methane (CH₄) and oxygen (O₂), and wherein the reactor is characterized by a reaction temperature of from about 700° C. to about 1,100° C.; (b) allowing at least a portion of the reactant mixture to react via an oxidative coupling of CH₄ reaction to form a product mixture, wherein the product mixture comprises primary products and unreacted methane, wherein the primary products comprise C₂₊ hydrocarbons and synthesis gas, wherein the C₂₊ hydrocarbons comprise olefins, and wherein a selectivity to primary products is from about 70% to about 99%; and (c) recovering at least a portion of the product mixture from the reactor. In an embodiment, the reactor can exclude a catalyst (e.g., a catalyst that catalyzes an oxidative coupling of CH₄ reaction).

Other than in the operating examples or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an embodiment,” “another embodiment,” “other embodiments,” “some embodiments,” and so forth, means that a particular element (e.g., feature, structure, property, and/or characteristic) described in connection with the embodiment is included in at least an embodiment described herein, and may or may not be present in other embodiments. In addition, it is to be understood that the described element(s) can be combined in any suitable manner in the various embodiments.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art.

Compounds are described herein using standard nomenclature. For example, any position not substituted by any indicated group is understood to have its valency filled by a bond as indicated, or a hydrogen atom. A dash (“—”) that is not between two letters or symbols is used to indicate a point of attachment for a substituent. For example, —CHO is attached through the carbon of the carbonyl group.

In an embodiment, a method for producing olefins and synthesis gas can comprise introducing a reactant mixture to a reactor, wherein the reactant mixture comprises methane (CH₄) and oxygen (O₂), and wherein the reactor can exclude a catalyst (e.g., a catalyst that catalyzes an oxidative coupling of CH₄ (OCM) reaction; an OCM catalyst); and allowing at least a portion of the reactant mixture to react via an OCM reaction to form a product mixture.

The OCM has been the target of intense scientific and commercial interest for more than thirty years due to the tremendous potential of such technology to reduce costs, energy, and environmental emissions in the production of ethylene (C₂H₄). As an overall reaction, in the OCM, CH₄ and O₂ react exothermically to form C₂H₄, water (H₂O) and heat.

Oxidation of methane at high temperatures (e.g., from about 700° C. to about 1,100° C.) can lead to the appearance of the following reactions, as shown in reactions (1)-(4):

2CH₄+O₂═C₂H₄+2H₂O, ΔH=−67 kcal/mol (1)

2CH₄+½O₂═C₂H₆+H₂O, ΔH=−42 kcal/mol (2)

CH₄+1.5O₂═CO+2H₂O, ΔH=−124 kcal/mol (3)

CH₄+2O₂═CO₂+2H₂O ΔH=−192 kcal/mol (4)

Generally, in the absence of an OCM catalyst, conversion of methane is low and the main products of conversion are CO and CO₂, as thermodynamically favored by reactions (3) and (4). Some of the best reported OCM outcomes encompass a ˜20% conversion of methane and ˜80% selectivity to desired C₂ hydrocarbons.

In an embodiment, a method for producing olefins and synthesis gas as disclosed herein can comprise conducting an OCM reaction in the absence of an OCM catalyst, by controlling a range of reaction temperature, a reactor residence time and a reactor feed composition (e.g., reactant mixture composition) in such a way to maximize a C₂₊ selectivity and the production of a synthesis gas with a high H₂/CO ratio (e.g., from about 0.5:1 to about 2:1), thereby minimizing CO₂ formation by reaction (4), as will be described in more detail later herein. In some embodiments, controlling a reactor feed composition can further comprise introducing to the reactor other components (e.g., reagents other than methane and oxygen), such as for example hydrogen, thereby changing the pathway of methane conversion reactions, as will be described in more detail later herein.

In an embodiment, the reactant mixture can comprise a hydrocarbon or mixtures of hydrocarbons, and oxygen. In some embodiments, the hydrocarbon or mixtures of hydrocarbons can comprise natural gas (e.g., CH₄), liquefied petroleum gas comprising C₂-C₅ hydrocarbons, C₆₊ heavy hydrocarbons (e.g., C₆ to C₂₄ hydrocarbons such as diesel fuel, jet fuel, gasoline, tars, kerosene, etc.), oxygenated hydrocarbons, biodiesel, alcohols, dimethyl ether, and the like, or combinations thereof. In an embodiment, the reactant mixture can comprise CH₄ and O₂.

In an embodiment, the O₂ used in the reaction mixture can be oxygen gas (which may be obtained via a membrane separation process), technical oxygen (which may contain some air), air, oxygen enriched air, or combinations thereof.

In an embodiment, the reactant mixture can be a gaseous mixture. In an embodiment, the reactant mixture can be characterized by a CH₄/O₂ molar ratio of from about 2:1 to about 40:1, alternatively from about 5:1 to about 30:1, alternatively from about 10:1 to about 25:1, alternatively from about 12:1 to about 20:1, or alternatively from about 14:1 to about 18:1.

In an embodiment, the reactant mixture can further comprise a diluent. The diluent is inert with respect to the OCM reaction, e.g., the diluent does not participate in the OCM reaction.

In an embodiment, the diluent can comprise water, nitrogen, inert gases, and the like, or combinations thereof. In an embodiment, the diluent can be present in the reactant mixture in an amount of from about 0.5% to about 80%, alternatively from about 5% to about 50%, or alternatively from about 10% to about 30%, based on the total volume of the reactant mixture.

In an embodiment, the reactant mixture can further comprise hydrogen (H₂). Without wishing to be limited by theory, introducing hydrogen to the reactant mixture can generate active species (e.g., active radical species), for example by interaction with oxygen, which can further generate new routes for the OCM reaction in the absence of an OCM catalyst. Generally, a stoichiometric equation reaction of hydrogen with oxygen can be described by reaction (5):

2H₂+O₂=2H₂O (5)

Further, without wishing to be limited by theory, at high reaction temperatures (e.g., from about 700° C. to about 1,100° C.), hydrogen and oxygen can create hydroxyl radicals and can propagate an OCM reaction in the presence of methane according to reactions (6)-(9):

H₂+O₂=2OH. (6)

OH.+CH₄=H₂O+CH₃. (7)

CH₃.+O₂═CH₃O₂. (8)

CH₃O₂.═CH₂O+OH. (9)

Without wishing to be limited by theory, hydroxyl radical groups (e.g., OH.) as produced by reaction (6) can abstract hydrogen from methane as shown in reaction (7), which can generate radical active species (e.g., CH₃.) for propagating the OCM reaction similarly to the generation of catalytic active species on a catalyst surface. Reaction (8) can significantly reduce C₂ selectivity. Further, without wishing to be limited by theory, addition of hydrogen to the reactant mixture can (i) generate radicals by reaction (6) and (ii) consume oxygen, thereby decreasing the role of reaction (8).

In an embodiment, the reactant mixture can be characterized by a CH₄/H₂ molar ratio of from about 10:1 to about 100:1, alternatively from about 10:1 to about 50:1, alternatively from about 10:1 to about 20:1, or alternatively from about 8:1 to about 15:1. In an embodiment, a hydrogen molar concentration in the reactant mixture does not exceed a methane molar concentration in the reactant mixture.

In an embodiment, the reactant mixture can be characterized by an O₂/H₂ molar ratio of from about 2:1 to about 10:1, alternatively from about 3:1 to about 9:1, or alternatively from about 5:1 to about 8:1.

In an embodiment, the reactant mixture can be characterized by a (CH₄+H₂)/0₂ molar ratio of from about 2:1 to about 40:1, alternatively from about 3:1 to about 25:1, alternatively from about 3:1 to about 16:1, alternatively from about 4:1 to about 12:1, or alternatively from about 4:1 to about 8:1.

In an embodiment, a method for producing olefins and synthesis gas can comprise introducing the reactant mixture to a reactor comprising a catalyst. In such embodiment, the reactor can comprise an isothermal reactor, a fluidized sand bath reactor, an autothermal reactor, an adiabatic reactor, a tubular reactor, a cooled tubular reactor, a continuous flow reactor, a reactor lined with an inert refractory material, a glass lined reactor, a ceramic lined reactor, and the like, or combinations thereof. In an embodiment, the inert refractory material can comprise silica, alumina, silicon carbide, boron nitride, titanium oxide, mullite, mixtures of oxides, and the like, or combinations thereof.

In an embodiment, the isothermal reactor can comprise a tubular reactor, a cooled tubular reactor, a continuous flow reactor, and the like, or combinations thereof.

In an embodiment, the isothermal reactor can comprise a reactor vessel located inside a fluidized sand bath reactor, wherein the fluidized sand bath provides isothermal conditions (i.e., substantially constant temperature) for the reactor. In such embodiment, the fluidized sand bath reactor can be a continuous flow reactor comprising an outer jacket comprising a fluidized sand bath. The fluidized sand bath can exchange heat with the reactor, thereby providing isothermal conditions for the reactor. Generally, a fluidized bath employs fluidization of a mass of finely divided inert particles (e.g., sand particles, metal oxide particles, aluminum oxide particles, metal oxides microspheres, quartz sand microspheres, aluminum oxide microspheres, silicon carbide microspheres) by means of an upward stream of gas, such as for example air, nitrogen, etc.

In an embodiment, the isothermal conditions can be provided by fluidization of heated microspheres around the isothermal reactor comprising the catalyst bed, wherein the microspheres can be heated at a temperature of from about 675° C. to about 1,100 ° C., alternatively from about 700° C. to about 1,050° C., or alternatively from about 750° C. to about 1,000° C.; and wherein the microspheres can comprise sand, metal oxides, quartz sand, aluminum oxide, silicon carbide, and the like, or combinations thereof. In an embodiment, the microspheres (e.g., inert particles) can have a size of from about 10 mesh to about 400 mesh, alternatively from about 30 mesh to about 200 mesh, or alternatively from about 80 mesh to about 100 mesh, based on U.S. Standard Sieve Size.

While in a fluidized state, the individual inert particles become microscopically separated from each other by the upward moving stream of gas. Generally, a fluidized bath behaves remarkably like a liquid, exhibiting characteristics which are generally attributable to a liquid state (e.g., a fluidized bed can be agitated and bubbled; inert particles of less density will float while those with densities greater than the equivalent fluidized bed density will sink; heat transfer characteristics between the fluidized bed and a solid interface can have an efficiency approaching that of an agitated liquid; etc.).

In an embodiment, isothermal conditions can be provided by fluidized aluminum oxide, such as for example by a BFS high temperature furnace, which is a high temperature calibration bath, and which is commercially available from Techne Calibration.

In an embodiment, the reaction mixture can be introduced to the reactor at a temperature of from about 150° C. to about 300° C., alternatively from about 175° C. to about 250° C., or alternatively from about 200° C. to about 225° C. As will be appreciated by one of skill in the art, and with the help of this disclosure, while the OCM reaction is exothermic, heat input is necessary for promoting the formation of methyl radicals from CH₄, as the C—H bonds of CH₄ are very stable, and the formation of methyl radicals from CH₄ is endothermic. In an embodiment, the reaction mixture can be introduced to the reactor at a temperature effective to promote an OCM reaction.

In an embodiment, the reactor can be characterized by a reaction temperature of from about 700° C. to about 1,100° C., alternatively from about 750° C. to about 1,050° C., alternatively from about 800° C. to about 1,025° C., or alternatively from about 950° C. to about 1,000° C.

In an embodiment, the diluent can provide for heat control of the OCM reaction, e.g., the diluent can act as a heat sink. Generally, an inert compound (e.g., a diluent) can absorb some of the heat produced in the exothermic OCM reaction, without degrading or participating in any reaction (OCM or other reaction), thereby providing for controlling a temperature inside the reactor. As will be appreciated by one of skill in the art, and with the help of his disclosure, the diluent can be introduced to the reactor at ambient temperature, or as part of the reaction mixture (at a reaction mixture temperature), and as such the temperature of the diluent entering the rector is much lower than the reaction temperature, and the diluent can act as a heat sink.

In an embodiment, the reactor can be characterized by a pressure of from about ambient pressure (e.g., atmospheric pressure) to about 500 psig, alternatively from about ambient pressure to about 200 psig, or alternatively from about ambient pressure to about 100 psig. In an embodiment, the method for producing olefins as disclosed herein can be carried out at ambient pressure.

In an embodiment, the reactor can be characterized by a residence time of from about 100 milliseconds (ms) to about 30 seconds (s), alternatively from about 150 ms to about 2 s, alternatively from about 300 ms to about 1 s, or alternatively from about 250 ms to about 750 ms. Generally, the residence time of a reactor refers to the average amount of time that a compound (e.g., a molecule of that compound) spends in that particular reactor, e.g., the average amount of time that it takes for a compound (e.g., a molecule of that compound) to travel through the reactor.

In an embodiment, the reactor can be characterized by a gas hourly space velocity (GHSV) of from about 30 h⁻¹ to about 20,000 h⁻¹, alternatively from about 1,000 h⁻¹ to about 17,500 h⁻¹, or alternatively from about 5,000 h⁻¹ to about 15,000 h⁻¹. Generally, the GHSV relates a reactant (e.g., reactant mixture) gas flow rate to a reactor volume. GHSV is usually measured at standard temperature and pressure.

In an embodiment, a method for producing olefins and synthesis gas can comprise allowing at least a portion of the reactant mixture to react via an oxidative coupling of CH₄ reaction to form a product mixture, wherein the product mixture comprises primary products and unreacted methane, wherein the primary products comprise C₂₊ hydrocarbons and synthesis gas, wherein the C₂₊ hydrocarbons comprise olefins, and wherein a selectivity to primary products can be from about 70% to about 99%.

Generally, a selectivity to a desired product or products refers to how much desired product was formed divided by the total products formed, both desired and undesired. For purposes of the disclosure herein, the selectivity to a desired product is a % selectivity based on moles converted into the desired product. Further, for purposes of the disclosure herein, a C, selectivity (e.g., C₂ selectivity, C₂₊ selectivity, etc.) can be calculated by dividing a number of moles of carbon (C) from CH₄ that were converted into the desired product (e.g., C_{C2H4}, C_{C2H6}, etc.) by the total number of moles of C from CH₄ that were converted (e.g., C_{C2H4}, C_{C2H6}, C_{C2H2}, C_{C3H6}, C_{C3H8}, C_{C4s}, C_{CO2}, C_{CO}, etc.). C_{C2H4}=number of moles of C from CH₄ that were converted into C₂H₄; C_{C2H6}=number of moles of C from CH₄ that were converted into C₂H₆; C_{C2H2}=number of moles of C from CH₄ that were converted into C₂H₂; C_{C3H6}=number of moles of C from CH₄ that were converted into C₃H₆; C_{C3H8}=number of moles of C from CH₄ that were converted into C₃H₈; C_{C4s}=number of moles of C from CH₄ that were converted into C₄ hydrocarbons (C₄s); C_{CO2}=number of moles of C from CH₄ that were converted into CO₂; C_{CO}=number of moles of C from CH₄ that were converted into CO; etc.

In an embodiment, the product mixture comprises coupling products, partial oxidation products (e.g., partial conversion products, such as CO, H₂, CO₂), and unreacted methane. In an embodiment, the coupling products can comprise olefins (e.g., alkenes, characterized by a general formula CₙH₂ₙ) and paraffins (e.g., alkanes, characterized by a general formula CₙH₂ₙ₊₂).

In an embodiment, the product mixture can comprise C₂₊ hydrocarbons and synthesis gas, wherein the C₂₊ hydrocarbons can comprise C₂ hydrocarbons and C₃ hydrocarbons. In an embodiment, the C₂₊ hydrocarbons can further comprise C₄ hydrocarbons (C₄s), such as for example butane, iso-butane, n-butane, butylene, etc. In some embodiments, the product mixture can comprise C₂H₄, C₂H₆, CH₄, CO, H₂, CO₂ and H₂O.

In an embodiment, the C₂ hydrocarbons can comprise ethylene (C₂H₄) and ethane (C₂H₆). In some embodiments, a C₂H₄ content of the product mixture can be higher than a C₂H₆ content of the product mixture. In an embodiment, the C₂ hydrocarbons can further comprise acetylene (C₂H₂).

In an embodiment, the C₃ hydrocarbons can comprise propylene (C₃H₆). In an embodiment, the C₃ hydrocarbons can further comprise propane (C₃H₈).

In an embodiment, a selectivity to primary products (e.g., Cₚₚ selectivity) can be from about 70% to about 99%, alternatively from about 80% to about 99%, alternatively from about 90% to about 99%, alternatively from about 75% to about 95%, or alternatively from about 80% to about 90%. The Cₚₚ selectivity refers to how much primary products (e.g., desired products, such as C₂ hydrocarbons, C₃ hydrocarbons, C₄s, CO for synthesis gas, etc.) were formed divided by the total products formed, including C₂H₄, C₃H₆, C₂H₆, C₃H₈, C₂H₂, C₄s, CO₂ and CO. For example, the Cₚₚ selectivity can be calculated by using equation (10):

$${C}_{pp} selectivity = \frac{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s} + {C}_{CO}\end{matrix}}{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s} + {C}_{{CO}_{2}} + {C}_{CO}\end{matrix}} \times 100 \% \qquad ( 10 )$$

As will be appreciated by one of skill in the art, if a specific product and/or hydrocarbon product is not produced in a certain OCM reaction/process, then the corresponding C_{Cx} is 0, and the term is simply removed from selectivity calculations.

In an embodiment, a selectivity to ethylene (C₂₌ selectivity) can be from about 10% to about 60%, alternatively from about 15% to about 55%, alternatively from about 20% to about 50%, or alternatively from about 50% to about 65%. The C₂₌ selectivity refers to how much C₂H₄ was formed divided by the total products formed, including C₂H₄, C₃H₆, C₂H₆, C₃H₈, C₂H₂, C₄s, CO₂ and CO. For example, the selectivity to ethylene can be calculated by using equation (11):

$${C}_{2 =} selectivity = \frac{2 {C}_{{C}_{2} {H}_{4}}}{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s} + {C}_{{CO}_{2}} + {C}_{CO}\end{matrix}} \times 100 \% \qquad ( 11 )$$

In an embodiment, a selectivity to C₂ hydrocarbons (C₂ selectivity) can be from about 10% to about 70%, alternatively from about 15% to about 65%, or alternatively from about 20% to about 60%. The C₂ selectivity refers to how much C₂H₄, C₂H₆, and C₂H₂ were formed divided by the total products formed, including C₂H₄, C₃H₆, C₂H₆, C₃H₈, C₂H₂, C₄s, CO₂ and CO. For example, the C₂ selectivity can be calculated by using equation (12):

$${C}_{2} selectivity = \frac{2 {C}_{{C}_{2} {H}_{4}} + 2 {C}_{{C}_{2} {H}_{6}} + 2 {C}_{{C}_{2} {H}_{2}}}{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s} + {C}_{{CO}_{2}} + {C}_{CO}\end{matrix}} \times 100 \% \qquad ( 12 )$$

In an embodiment, a selectivity to C₂₋ hydrocarbons (C₂₊ selectivity) can be from about 15% to about 75%, alternatively from about 20% to about 70%, or alternatively from about 20% to about 65%. The C₂₊ selectivity refers to how much C₂H₄, C₃H₆, C₂H₆, C₃H₈, C₂H₂, and C₄s were formed divided by the total products formed, including C₂H₄, C₃H₆, C₂H₆, C₃H₈, C₂H₂, C₄s, CO₂ and CO. For example, the C₂₊ selectivity can be calculated by using equation (13):

$${C}_{2 +} selectivity = \frac{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s}\end{matrix}}{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s} + {C}_{{CO}_{2}} + {C}_{CO}\end{matrix}} \times 100 \% \qquad ( 13 )$$

In an embodiment, a methane conversion can be from about 5% to about 25%, alternatively from about 7.5% to about 22.5%, or alternatively from about 10% to about 20%. Generally, a conversion of a reagent or reactant refers to the percentage (usually mol %) of reagent that reacted to both undesired and desired products, based on the total amount (e.g., moles) of reagent present before any reaction took place. For purposes of the disclosure herein, the conversion of a reagent is a % conversion based on moles converted. For example, the methane conversion can be calculated by using equation (14):

$${CH}_{4} conversion = \frac{{C}_{{CH}_{4}}^{in} - {C}_{{CH}_{4}}^{out}}{{C}_{{CH}_{4}}^{in}} \times 100 \% \qquad ( 14 )$$

wherein C_{CH}_{₄}ⁱⁿ=number of moles of C from CH₄ that entered the reactor as part of the reactant mixture; and C_{CH}^{out}=number of moles of C from CH₄ that was recovered from the reactor as part of the product mixture.

In an embodiment, a sum of CH₄ conversion plus the selectivity to C₂₊ hydrocarbons can be equal to or greater than about 100%, alternatively equal to or greater than about 105%, or alternatively equal to or greater than about 110%. As will be appreciated by one of skill in the art, and with the help of this disclosure, the lower the residence time, the higher the selectivity to desired products, and the lower the methane conversion. Further, as will be appreciated by one of skill in the art, and with the help of this disclosure, the higher the reaction temperature, the higher the selectivity to desired products (e.g., olefins, hydrocarbons, etc.); however, generally, extremely high reaction temperatures (e.g., over about 1,100° C.) can lead to an increase in deep oxidation products (e.g., CO, CO₂).

In embodiments where hydrogen is present in the reactant mixture, the methane conversion and/or C₂₊ selectivity in an OCM reaction as disclosed herein can be increased when compared to a methane conversion and/or C₂₊ selectivity in an otherwise similar OCM reaction lacking H₂ in the reactant mixture. For example, methane conversion can be increased by equal to or greater than about 5%, alternatively equal to or greater than about 10%, or alternatively equal to or greater than about 15%, when compared to a methane conversion in an otherwise similar oxidative coupling of methane reaction conducted with a reactant mixture lacking hydrogen. Further, for example, a selectivity to C₂₊ hydrocarbons can be increased by equal to or greater than about 5%, alternatively equal to or greater than about 10%, or alternatively equal to or greater than about 15%, when compared to a C₂₊ selectivity in an otherwise similar oxidative coupling of methane reaction conducted with a reactant mixture lacking hydrogen.

In an embodiment, a method for producing olefins and synthesis gas as disclosed herein can further comprise minimizing deep oxidation of methane to CO₂. In an embodiment, the product mixture can comprise less than about 15 mol % CO₂, alternatively less than about 10 mol % CO₂, or alternatively less than about 5 mol % CO₂.

In an embodiment, equal to or greater than about 2 mol %, alternatively equal to or greater than about 5 mol %, or alternatively equal to or greater than about 10 mol % of the reactant mixture can be converted to olefins.

In an embodiment, equal to or greater than about 2 mol %, alternatively equal to or greater than about 5 mol %, or alternatively equal to or greater than about 10 mol % of the reactant mixture can be converted to ethylene.

In an embodiment, equal to or greater than about 4 mol %, alternatively equal to or greater than about 8 mol %, or alternatively equal to or greater than about 12 mol % of the reactant mixture can be converted to C₂ hydrocarbons.

In an embodiment, equal to or greater than about 5 mol %, alternatively equal to or greater than about 10 mol %, or alternatively equal to or greater than about 15 mol % of the reactant mixture can be converted to C₂₊ hydrocarbons.

In an embodiment, equal to or greater than about 10 mol %, alternatively equal to or greater than about 15 mol %, or alternatively equal to or greater than about 20 mol % of the reactant mixture can be converted to synthesis gas. Generally, in industrial settings, synthesis gas is produced by an endothermic process of steam reforming of natural gas. In an embodiment, the synthesis gas can be produced as disclosed herein as a side reaction in an OCM reaction/process.

In an embodiment, the product mixture can comprise synthesis gas (e.g., CO and H₂). In an embodiment, at least a portion of the H₂ found in the product mixture can be produced by the OCM reaction. Synthesis gas, also known as syngas, is generally a gas mixture consisting primarily of CO and H₂, and sometimes CO₂. Synthesis gas can be used for producing olefins; for producing methanol; for producing ammonia and fertilizers; in the steel industry; as a fuel source (e.g., for electricity generation); etc. In such embodiment, the product mixture (e.g., the synthesis gas of the product mixture) can be characterized by a hydrogen (H₂) to carbon monoxide (CO) ratio of from about 0.5:1 to about 2:1, alternatively from about 0.7:1 to about 1.8:1, or alternatively from about 1:1 to about 1.75:1.

In an embodiment, a selectivity to CO (Cₒₒ selectivity) can be from about 25% to about 85%, alternatively from about 30% to about 82.5%, or alternatively from about 40% to about 80%. The C_{CO} selectivity refers to how much CO was formed divided by the total products formed, including C₂H₄, C₃H₆, C₂H₆, C₃H₈, C₂H₂, Cₒs, CO₂ and CO. For example, the C_{CO} selectivity can be calculated by using equation (15):

$${C}_{CO} selectivity = \frac{{C}_{CO}}{\begin{matrix}2 {C}_{{C}_{2} {H}_{4}} + 2 {C}_{{C}_{2} {H}_{6}} + 2 {C}_{{C}_{2} {H}_{2}} + \\ 3 {C}_{{C}_{3} {H}_{6}} + 3 {C}_{{C}_{3} {H}_{8}} + 4 {C}_{{C}_{4} s} + {C}_{{CO}_{2}} + {C}_{CO}\end{matrix}} \times 100 \% \qquad ( 15 )$$

In an embodiment, at least a portion of the synthesis gas can be separated from the product mixture to yield recovered synthesis gas, for example by cryogenic distillation. As will be appreciated by one of skill in the art, and with the help of this disclosure, the recovery of synthesis gas is done as a simultaneous recovery of both H₂ and CO.

In an embodiment, at least a portion of the recovered synthesis gas can be further converted to olefins. For example, the recovered synthesis gas can be converted to alkanes by using a Fisher-Tropsch process, and the alkanes can be further converted by dehydrogenation into olefins.

In an embodiment, at least a portion of the unreacted methane and at least a portion of the synthesis gas can be separated from the product mixture to yield a recovered synthesis gas mixture, wherein the recovered synthesis gas mixture comprises CO, H₂, and CH₄. In an embodiment, at least a portion of the recovered synthesis gas mixture can be further converted to olefins. In some embodiments, at least a portion of the recovered synthesis gas mixture can be further used as fuel to generate power. In other embodiments, at least a portion of the unreacted methane can be recovered and recycled to the reactant mixture.

In an embodiment, at least a portion of the recovered synthesis gas mixture can be further converted to liquid hydrocarbons (e.g., alkanes) by a Fisher-Tropsch process. In such embodiment, the liquid hydrocarbons can be further converted by dehydrogenation into olefins.

In some embodiments, at least a portion of the recovered synthesis gas mixture can be further converted to methane via a methanation process.

In an embodiment, a method for producing olefins and synthesis gas can comprise recovering at least a portion of the product mixture from the reactor, wherein the product mixture can be collected as an outlet gas mixture from the reactor. In an embodiment, the product mixture can comprise primary products and unreacted methane, wherein the primary products comprise C₂₊ hydrocarbons and synthesis gas, and wherein the C₂₊ hydrocarbons comprise olefins. In an embodiment, a method for producing olefins and synthesis gas can comprise recovering at least a portion of the olefins and/or at least a portion of the synthesis gas from the product mixture.

In an embodiment, the product mixture can comprise C₂₊ hydrocarbons (including olefins), unreacted methane, synthesis gas and optionally a diluent. When water (e.g., steam) is used as a diluent, the water can be separated from the product mixture prior to separating any of the other product mixture components. For example, by cooling down the product mixture to a temperature where the water condenses (e.g., below 100° C. at ambient pressure), the water can be removed from the product mixture, by using a flash chamber for example.

In an embodiment, at least a portion of the C₂₊ hydrocarbons can be separated (e.g., recovered) from the product mixture to yield recovered C₂₊ hydrocarbons. The C₂₊ hydrocarbons can be separated from the product mixture by using any suitable separation technique. In an embodiment, at least a portion of the C₂₊ hydrocarbons can be separated from the product mixture by distillation (e.g., cryogenic distillation).

In an embodiment, at least a portion of the recovered C₂₊ hydrocarbons can be used for ethylene production. In some embodiments, at least a portion of ethylene can be separated from the recovered C₂₊ hydrocarbons to yield recovered ethylene, by using any suitable separation technique (e.g., distillation). In other embodiments, at least a portion of the recovered C₂₊ hydrocarbons can be converted to ethylene, for example by a conventional steam cracking process.

In an embodiment, at least a portion of the unreacted methane can be separated from the product mixture to yield recovered methane. Methane can be separated from the product mixture by using any suitable separation technique, such as for example distillation (e.g., cryogenic distillation). In an embodiment, at least a portion of the recovered methane can be recycled to the reactant mixture.

In an embodiment, the reactor can be a multi-stage reactor, wherein the multi-stage reactor can comprise multiple stages of reaction (e.g., OCM reaction). In an embodiment, the multi-stage reactor can comprise from about 2 to about 10 reactors in series, alternatively from about 3 to about 8 reactors in series, or alternatively from about 4 to about 6 reactors in series. In an embodiment, the multi-stage reactor can comprise any suitable number and arrangement of reactors (e.g., stages, reaction stages) in series and/or in parallel to achieve a desired methane conversion and selectivity to desired products. In an embodiment, a selectivity to desired products obtained from a multi-stage reactor as disclosed herein can be higher than a selectivity to desired products obtained from a single stage reactor as disclosed herein.

In some embodiments, the multi-stage reactor can comprise one initial stage reactor, at least one intermediate stage reactor, and one finishing stage reactor. As will be appreciated by one of skill in the art, and with the help of this disclosure, the initial stage reactor, the intermediate stage reactor and the finishing stage reactor can each individually comprise any suitable number and arrangement of reactors (e.g., stages, reaction stages) in series and/or in parallel to achieve a desired methane conversion and selectivity to desired products.

In an embodiment, an initial stage reactant mixture can be introduced to an initial stage reactor, wherein the initial stage reactant mixture can comprise methane, oxygen and optionally hydrogen. In an embodiment, an intermediate stage reactant mixture can be introduced to an intermediate stage reactor, wherein the intermediate stage reactant mixture can comprise oxygen and optionally hydrogen. In an embodiment, a finishing stage reactant mixture can be introduced to a finishing stage reactor, wherein the finishing stage reactant mixture can comprise oxygen.

In an embodiment, the initial stage reactor and the at least one intermediate stage reactor can operate at partial oxygen conversion, wherein the oxygen conversion can be from equal to or greater than about 50% to equal to or less than about 99%, alternatively from equal to or greater than about 55% to equal to or less than about 95%, or alternatively from equal to or greater than about 60% to equal to or less than about 90%.

In an embodiment, near complete oxygen conversion can be achieved in the finishing stage reactor, e.g., oxygen conversion in the finishing stage reactor can be equal to or greater than about 99%, alternatively equal to or greater than about 99.5%, or alternatively equal to or greater than about 99.9%.

In an embodiment, a selectivity to C₂₊ hydrocarbons in a multi-stage reactor can be increased by equal to or greater than about 5%, alternatively by equal to or greater than about 10%, or alternatively by equal to or greater than about 15%, when compared to a selectivity to C₂₊ hydrocarbons of an otherwise similar oxidative coupling of methane reaction conducted in a single stage reactor.

In an embodiment, a synthesis gas H₂/CO molar ratio produced by a multi-stage reactor as disclosed herein can be equal to or greater than about 1.0, alternatively equal to or greater than about 1.5, alternatively equal to or greater than about 1.9, or alternatively equal to or greater than about 2.

In an embodiment, the synthesis gas H₂/CO molar ratio produced by a multi-stage reactor can be increased by equal to or greater than about 25%, alternatively equal to or greater than about 50%, or alternatively equal to or greater than about 100%, when compared to a synthesis gas H₂/CO molar ratio produced by an otherwise similar oxidative coupling of methane reaction conducted in a single stage reactor.

In an embodiment, a method for producing ethylene and synthesis gas can comprise (a) introducing a reactant mixture to a reactor, wherein the reactor can exclude a catalyst, wherein the reactant mixture can comprise methane (CH₄) and oxygen (O₂), and wherein the reactor can be characterized by a reaction temperature of from about 700° C. to about 1,100° C.; (b) allowing at least a portion of the reactant mixture to react via an oxidative coupling of CH₄ reaction to form a product mixture, wherein the product mixture can comprise primary products and unreacted methane, wherein the primary products can comprise C₂₊ hydrocarbons and synthesis gas, wherein the C₂₊ hydrocarbons can comprise ethylene, and wherein a selectivity to primary products can be from about 70% to about 99%; (c) recovering at least a portion of the product mixture from the reactor; (d) separating at least a portion of ethylene from the product mixture to yield recovered ethylene; and (e) separating at least a portion of the synthesis gas from the product mixture to yield recovered synthesis gas.

In an embodiment, a method for producing ethylene and synthesis gas can comprise (a) introducing a reactant mixture to a reactor, wherein the reactant mixture can comprise methane (CH₄), oxygen (O₂) and hydrogen (H₂), wherein the reactant mixture can be characterized by a CH₄/O₂ molar ratio of from about 14:1 to about 18:1, wherein the reactant mixture can be characterized by a CH₄/H₂ molar ratio of from about 8:1 to about 15:1, wherein the reactant mixture can be characterized by a O₂/H₂ molar ratio of from about 5:1 to about 8:1, wherein the reactor can be characterized by a reaction temperature of from about 950° C. to about 1,000° C., wherein the reactor can be characterized by a residence time of from about 250 milliseconds to about 750 milliseconds, and wherein the reactor can exclude a catalyst; (b) allowing at least a portion of the reactant mixture to react via an oxidative coupling of CH₄ reaction to form a product mixture, wherein the product mixture can comprise primary products and unreacted methane, wherein the primary products can comprise C₂₊ hydrocarbons and synthesis gas, wherein the C₂₊ hydrocarbons can comprise ethylene, and wherein a selectivity to primary products is from about 90% to about 99%; (c) recovering at least a portion of the product mixture from the reactor; (d) separating at least a portion of the ethylene from the product mixture by cryogenic distillation to yield recovered ethylene; and (d) separating at least a portion of the synthesis gas from the product mixture by cryogenic distillation to yield recovered synthesis gas.

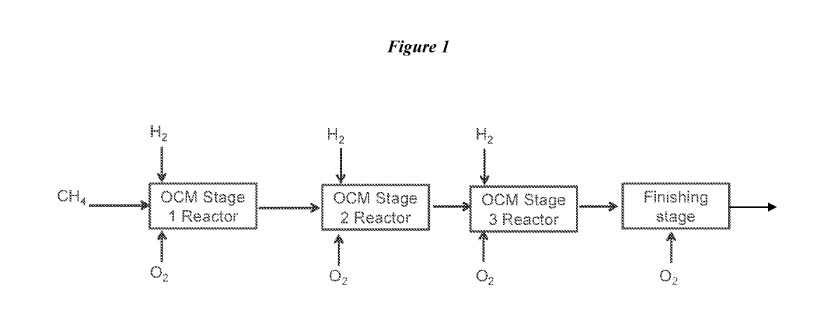

In an embodiment, a method for producing ethylene and synthesis gas can comprise introducing a reactant mixture to a reactor, wherein the reactor can exclude a catalyst, wherein the reactor can be characterized by a reaction temperature of from about 700° C. to about 1,100° C., and wherein the reactor can be a multi-stage reactor as represented in FIG. 1. Referring to the embodiment of FIG. 1, the multi-stage reactor can comprise an initial stage reactor (e.g., OCM stage 1 reactor); two intermediate stage reactors (e.g., OCM stage 2 reactor and OCM stage 3 reactor); and a finishing stage reactor. An initial stage reactant mixture can be introduced to the initial stage reactor, wherein the initial stage reactant mixture can comprise methane, oxygen and hydrogen. In an embodiment, at least a portion of the initial stage reactant mixture can be allowed to react via an oxidative coupling of CH₄ reaction to form an initial stage product mixture, wherein at least a portion of the initial stage product mixture can be communicated to the OCM stage 2 reactor. H₂ and O₂ can be introduced to the OCM stage 2 reactor, and at least a portion of the initial stage product mixture, O₂ and H₂ can be allowed to react via an OCM reaction to form a second stage product mixture, wherein at least a portion of the second stage product mixture can be communicated to the OCM stage 3 reactor. H₂ and O₂ can be introduced to the OCM stage 3 reactor, and at least a portion of the second stage product mixture, O₂ and H₂ can be allowed to react via an OCM reaction to form a third stage product mixture, wherein at least a portion of the third stage product mixture can be communicated to the finishing stage reactor. O₂ can be introduced to the finishing stage reactor, and at least a portion of the third stage product mixture and O₂ can be allowed to react via an OCM reaction to form a product mixture (e.g., finishing stage product mixture), wherein at least a portion of the product mixture can be recovered from the finishing stage reactor. The product mixture recovered from the finishing stage reactor can comprise primary products and unreacted methane, wherein the primary products can comprise C₂₊ hydrocarbons and synthesis gas, wherein the C₂₊ hydrocarbons can comprise ethylene, and wherein a selectivity to primary products can be from about 70% to about 99%. The method for producing ethylene and synthesis gas in a multi-stage reactor can further comprise (i) separating at least a portion of ethylene from the product mixture to yield recovered ethylene; and (ii) separating at least a portion of the synthesis gas from the product mixture to yield recovered synthesis gas.

In an embodiment, a method for producing olefins (e.g., ethylene) and synthesis gas as disclosed herein can advantageously display improvements in one or more method characteristics when compared to an otherwise similar method conducted with a catalyst. Generally, in the absence of a catalyst, conversion of methane is low and the main products of conversion are CO and CO₂. While the catalyst can lead to the change of product distribution and increase of selectivity to C₂ products, there are several issues associated with the use of catalysts, such as for example loss of activity and selectivity after a very short time due to the sintering of active sites or evaporation of active materials from the catalyst. The method for producing olefins and synthesis gas as disclosed herein can advantageously control a distribution of products of OCM reaction by selection of the proper feed composition with addition of some components to the feed (e.g., hydrogen), which may change the pathway of methane conversion reactions, controlling temperature and residence time.

In an embodiment, the method for producing olefins and synthesis gas as disclosed herein can advantageously allow for the use of an increased reaction temperature (e.g., from about 700° C. to about 1,100° C.) when hydrogen is part of the reactant mixture, owing in part to elimination of some portion of deep oxidation reactions in the presence of hydrogen.

In an embodiment, the method for producing olefins and synthesis gas as disclosed herein at high temperatures (e.g., from about 700° C. to about 1,100° C.) and short residence times (e.g., from about 100 ms to about 30 s) can advantageously provide for high C₂₊ selectivity along with synthesis gas with high H₂/CO molar ratio (e.g., up to about 2:1), wherein the selectivity to primary products can be very high (e.g., up to about 99%). Additional advantages of the methods for the production of olefins (e.g., ethylene) and synthesis gas as disclosed herein can be apparent to one of skill in the art viewing this disclosure.

### EXAMPLES

The subject matter having been generally described, the following examples are given as particular embodiments of the disclosure and to demonstrate the practice and advantages thereof. It is understood that the examples are given by way of illustration and are not intended to limit the specification of the claims to follow in any manner.

### Example 1

Oxidative coupling of methane (OCM) reactions were conducted in the absence of a catalyst as follows. Methane, hydrogen and oxygen gases, along with an internal standard, an inert gas (neon) were fed to a quartz reactor with an internal diameter (I.D.) of 4 mm and were heated using a traditional clamshell furnace at a desired set point temperature. The reactor was first heated to a desired temperature under an inert gas flow and then a desired gas mixture was fed to the reactor.

Selectivities and conversions were calculated as outlined in equations (10)-(15), and the data are displayed in Table 1. The data in Table 1 were acquired in the absence of a catalyst, at a feed CH₄/O₂ molar ratio of 16:1; a reaction temperature of 975° C.; and a residence time of 512 ms.

TABLE 1

| | |
|---|---|
| % CH4 Conv. | 7.1 |
| % O2 Conv. | 66.9 |
| ‘C’ Selectivities | |
| C2= | 40.7 |
| C2 | 11.1 |
| C3= | 5.6 |
| C3 | 0.0 |
| % C2+ | 57.5 |
| % CO sel. | 40.4 |
| % CO2 sel. | 2.2 |
| H2/CO | 1.9 |
| ‘C’ BALANCE, % | 100.6 |

The data in Table 1 show that a high H₂/C0 molar ratio of 1.9:1 was achieved with a high selectivity of 57.5% towards C₂₊ hydrocarbons, making the total selectivity to primary (e.g., useful) products about ˜98%. The co-produced syngas composition is suitable for conversion to olefins by a Fisher-Tropsch process.

### Example 2

Oxidative coupling of methane (OCM) reactions were conducted in the absence of a catalyst under conditions as described in Example 1, but with different residence time and in a reactor of different dimensions: a 22 mm I.D. quartz reactor was used for acquiring the data in Example 2.

Selectivities and conversions were calculated as outlined in equations (10)-(15), and the data are displayed in Tables 2 and 3. The data in Tables 2 and 3 were acquired the absence of a catalyst.

The data in Table 2 were acquired in the absence of a catalyst, in a blank quartz tube with an inner diameter of 22 mm, at a feed (CH₄+H₂)/O₂ molar ratio of 10:1.

TABLE 2

| | | | |
|---|---|---|---|
| Feed Temp., C. | 675 | 675 | 675 |
| Residence time, s | 20.5 | 20.5 | 10.3 |
| H2% in CH4 feed | 0.0 | 4.0 | 4.0 |
| % CH4 Conv. | 8.8 | 10.5 | 8.7 |
| % O2 Conv. | 77.6 | 97.6 | 79.3 |
| % ‘C’ Selectivities | | | |
| C2= | 26.6 | 25.7 | 26.2 |
| C2 | 17.4 | 14.2 | 19.0 |
| C3= | 3.1 | 2.1 | 2.1 |
| C3 | 0.0 | 0.0 | 0.0 |
| C2+ | 47.1 | 42.0 | 47.2 |
| CO | 49.2 | 53.0 | 49.2 |
| CO2 | 3.8 | 5.0 | 3.6 |
| H2/CO ratio | 0.5 | 0.2 | 0.2 |
| ‘C’ BALANCE, % | 100.7 | 99.8 | 100.1 |

The data in Table 2 clearly show an increase in both methane and oxygen conversion, when hydrogen is added to the feed at same residence time. In the presence of hydrogen, the same levels of methane and oxygen conversions are observed at shorter contact times.

The data in Table 3 were acquired in the absence of a catalyst, in a quartz reactor with an inner diameter of 4 mm filled with quartz chips (18-35 mesh); at a feed (CH₄+H₂)/O₂ molar ratio of 16:1; a reaction temperature of 975° C.; and a residence time of 491 ms.

TABLE 3

| | | |
|---|---|---|
| H₂% in feed CH₄ | 4 | 0 |
| % CH₄ | 6.7 | 6.3 |
| % O₂ Conversion | 75.1 | 66.9 |
| ‘C’ Selectivities | | |
| C₂= | 41.7 | 40.7 |
| C₂ | 9.6 | 11.1 |
| C₃= | 4.5 | 5.6 |
| C₃ | 0.0 | 0.0 |
| C₂+ | 55.8 | 57.5 |
| CO | 42.0 | 40.4 |
| CO₂ | 2.2 | 2.2 |
| H₂/CO | 1.3 | 1.9 |

The data in Table 3 show higher conversion of methane and oxygen in thermal reaction with addition of H₂ in feed. The data in Tables 2 and 3 showed the positive effect of hydrogen addition in high temperature methane oxidative coupling reaction, leading to a reduction in residence time (to reach the same methane conversion) by up to 50%.

It was further established that the effect of hydrogen is very sensitive to the L/D parameter of the reactor, which is due to the performance of the chain reactions, which is in turn very sensitive to the L/D parameter of the reactor (L=length of reactor; D=inner diameter of reactor). For example, in the case of a quartz reactor comprising a 7 mm inner diameter quartz tube, addition of hydrogen led to an increase of methane conversion from 4.5% to 10% at 800° C. with an increase in C₂₊ selectivity from 40% to 45%; while in the case of a quartz reactor with a 12 mm inner diameter, the increase of methane conversion was from 9% to 15% with an increase in C₂₊ selectivity from 45% to 55%.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed herein are provided solely for their disclosure prior to the filing date of the present application. Nothing herein is to be construed as an admission that the inventors are not entitled to antedate such disclosure by virtue of prior invention.

In any application before the United States Patent and Trademark Office, the Abstract of this application is provided for the purpose of satisfying the requirements of 37 C.F.R. §1.72 and the purpose stated in 37 C.F.R. §1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

The present disclosure is further illustrated by the following examples, which are not to be construed in any way as imposing limitations upon the scope thereof. On the contrary, it is to be clearly understood that resort can be had to various other aspects, embodiments, modifications, and equivalents thereof which, after reading the description herein, can be suggest to one of ordinary skill in the art without departing from the spirit of the present invention or the scope of the appended claims.

### Additional Disclosure

A first aspect, which is a method for producing olefins and synthesis gas comprising (a) introducing a reactant mixture to a reactor, wherein the reactant mixture comprises methane (CH₄) and oxygen (O₂), wherein the reactor is characterized by a reaction temperature of from about 700° C. to about 1,100° C.; (b) allowing at least a portion of the reactant mixture to react via an oxidative coupling of CH₄ reaction to form a product mixture, wherein the product mixture comprises primary products and unreacted methane, wherein the primary products comprise C₂₊ hydrocarbons and synthesis gas, wherein the C₂₊ hydrocarbons comprise olefins, and wherein a selectivity to primary products is from about 70% to about 99%; and (c) recovering at least a portion of the product mixture from the reactor.

A second aspect, which is the method of the first aspect, wherein the reactor is characterized by a residence time of from about 100 milliseconds to about 30 seconds.

A third aspect, which is the method of any one of the first and the second aspects, wherein the reactor is characterized by a pressure of from about ambient pressure to about 500 psig.

A fourth aspect, which is the method of any one of the first through the third aspects, wherein the reactant mixture is characterized by a CH₄/O₂ molar ratio of from about 2:1 to about 40:1.

A fifth aspect, which is the method of any one of the first through the fourth aspects, wherein the reactant mixture further comprises hydrogen (H₂).

A sixth aspect, which is the method of the fifth aspect, wherein the reactant mixture is characterized by a CH₄/H₂ molar ratio of from about 10:1 to about 100:1.

A seventh aspect, which is the method of any one of the first through the sixth aspects, wherein the reactant mixture is characterized by an O₂/H₂ molar ratio of from about 2:1 to about 10:1.

An eighth aspect, which is the method of any one of the first through the seventh aspects, wherein the reactant mixture is characterized by a (CH₄+H₂)/O₂ molar ratio of from about 2:1 to about 40:1.

A ninth aspect, which is the method of any one of the first through the eighth aspects, wherein the reactor is characterized by a gas hourly space velocity of from about 30 h⁻¹ to about 20,000 h⁻¹.

A tenth aspect, which is the method of any one of the first through the ninth aspects, wherein the reactant mixture further comprises a diluent.

An eleventh aspect, which is the method of the tenth aspect, wherein the diluent comprises water, nitrogen, inert gases, or combinations thereof.

A twelfth aspect, which is the method of any one of the first through the eleventh aspects, wherein the reactor comprises an isothermal reactor, a fluidized sand bath reactor, an autothermal reactor, an adiabatic reactor, a tubular reactor, a cooled tubular reactor, a continuous flow reactor, a reactor lined with an inert refractory material, a glass lined reactor, a ceramic lined reactor, or combinations thereof.

A thirteenth aspect, which is the method of the twelfth aspect, wherein the inert refractory material comprises silica, alumina, silicon carbide, boron nitride, titanium oxide, mullite, mixtures of oxides, or combinations thereof.

A fourteenth aspect, which is the method of any one of the first through the thirteenth aspects, wherein the reactor excludes a catalyst.

A fifteenth aspect, which is the method of any one of the first through the fourteenth aspects, wherein a methane conversion is from about 5% to about 25%.

A sixteenth aspect, which is the method of any one of the first through the fifteenth aspects, wherein the C₂₊ hydrocarbons comprise C₂ hydrocarbons and C₃ hydrocarbons.

A seventeenth aspect, which is the method of the sixteenth aspects, wherein the C₂ hydrocarbons comprise ethylene and ethane.

An eighteenth aspect, which is the method of any one of the first through the seventeenth aspects, wherein the C₃ hydrocarbons comprise propylene.

A nineteenth aspect, which is the method of the eighteenth aspect, wherein the C₃ hydrocarbons further comprise propane.

A twentieth aspect, which is the method of any one of the first through the nineteenth aspects, wherein a selectivity to C₂₊ hydrocarbons is from about 15% to about 75%.

A twenty-first aspect, which is the method of any one of the first through the twentieth aspects, wherein a selectivity to C₂ hydrocarbons is from about 10% to about 70%.

A twenty-second aspect, which is the method the seventeenth aspect, wherein a selectivity to ethylene is from about 10% to about 60%.

A twenty-third aspect, which is the method of any one of the first through the twenty-second aspects, wherein equal to or greater than about 2 mol % of the reactant mixture is converted to olefins.

A twenty-fourth aspect, which is the method of any one of the first through the twenty-third aspects, wherein equal to or greater than about 2 mol % of the reactant mixture is converted to ethylene.

A twenty-fifth aspect, which is the method of any one of the first through the twenty-fourth aspects, wherein equal to or greater than about 4 mol % of the reactant mixture is converted to C₂ hydrocarbons.

A twenty-sixth aspect, which is the method of any one of the first through the twenty-fifth aspects, wherein equal to or greater than about 5 mol % of the reactant mixture is converted to C₂₊ hydrocarbons.

A twenty-seventh aspect, which is the method of any one of the first through the twenty-sixth aspects, wherein equal to or greater than about 10 mol % of the reactant mixture is converted to synthesis gas.

A twenty-eighth aspect, which is the method of any one of the first through the twenty-seventh aspects, wherein the synthesis gas is characterized by a hydrogen (H₂) to carbon monoxide (CO) molar ratio of from about 0.5:1 to about 2:1.

A twenty-ninth aspect, which is the method of any one of the first through the twenty-eighth aspects, wherein a selectivity to CO is from about 25% to about 85%.

A thirtieth aspect, which is the method of any one of the first through the twenty-ninth aspects, wherein at least a portion of the synthesis gas is separated from the product mixture to yield recovered synthesis gas.

A thirty-first aspect, which is the method of the thirtieth aspect, wherein at least a portion of the synthesis gas is separated from the product mixture by cryogenic distillation.

A thirty-second aspect, which is the method of any one of the first through the thirty-first aspects, wherein at least a portion of the recovered synthesis gas is further converted to olefins.

A thirty-third aspect, which is the method of any one of the first through the thirty-second aspects, wherein at least a portion of the synthesis gas and at least a portion of the unreacted methane are separated from the product mixture to yield a recovered synthesis gas mixture.

A thirty-fourth aspect, which is the method of the thirty-third aspect, wherein at least a portion of the recovered synthesis gas mixture is further converted to olefins.

A thirty-fifth aspect, which is the method of any one of the first through the thirty-fourth aspects, wherein at least a portion of the recovered synthesis gas mixture is further converted to liquid hydrocarbons by a Fischer-Tropsch process.

A thirty-sixth aspect, which is the method of any one of the first through the thirty-fifth aspects, wherein at least a portion of the recovered synthesis gas mixture is further used as fuel to generate power.

A thirty-seventh aspect, which is the method of any one of the first through the thirty-sixth aspects, wherein at least a portion of the C₂₊ hydrocarbons is separated from the product mixture to yield recovered C₂₊ hydrocarbons.

A thirty-eighth aspect, which is the method of the thirty-seventh aspect, wherein at least a portion of the recovered C₂₊ hydrocarbons is used for ethylene production.

A thirty-ninth aspect, which is the method of the thirty-eighth aspect further comprising separating at least a portion of the ethylene from the recovered C₂₊ hydrocarbons to yield recovered ethylene.

A fortieth aspect, which is the method of any one of the first through the thirty-ninth aspects further comprising converting at least a portion of the recovered C₂₊ hydrocarbons to ethylene.

A forty-first aspect, which is the method of any one of the first through the fortieth aspects, wherein at least a portion of the unreacted methane is separated from the product mixture to yield recovered methane.

A forty-second aspect, which is the method of the forty-first aspect, wherein at least a portion of the recovered methane is recycled to the reactant mixture.

A forty-third aspect, which is the method of any one of the first through the forty-second aspects, wherein the product mixture comprises less than about 15 mol % carbon dioxide (CO₂).

A forty-fourth aspect, which is the method of any one of the first through the forty-third aspects, wherein further comprising minimizing deep oxidation of methane to carbon dioxide (CO₂).

A forty-fifth aspect, which is the method of any one of the first through the forty-fourth aspects, wherein a methane conversion is increased by equal to or greater than about 5% when compared to an otherwise similar oxidative coupling of methane reaction conducted with a reactant mixture lacking hydrogen.

A forty-sixth aspect, which is the method of any one of the first through the forty-fifth aspects, wherein a selectivity to C₂₊ hydrocarbons is increased by equal to or greater than about 5% when compared to an otherwise similar oxidative coupling of methane reaction conducted with a reactant mixture lacking hydrogen.

A forty-seventh aspect, which is the method of the first aspect, wherein the reactor comprises from about 2 to about 10 reactors in series.

A forty-eighth aspect, which is the method of the forty-seventh aspect, wherein an initial stage reactant mixture comprising methane, oxygen and optionally hydrogen is introduced to an initial stage reactor.

A forty-ninth aspect, which is the method of the forty-eighth aspect, wherein an oxygen conversion in the initial stage reactor is from equal to or greater than about 50% to equal to or less than about 99%.

A fiftieth aspect, which is the method of any one of the forty-seventh through the forty-ninth aspects, wherein an intermediate stage reactant mixture comprising oxygen and optionally hydrogen is introduced to an intermediate stage reactor.

A fifty-first aspect, which is the method of the fiftieth aspect, wherein an oxygen conversion in the intermediate stage reactor is from equal to or greater than about 50% to equal to or less than about 99%.

A fifty-second aspect, which is the method of any one of the forty-seventh through the fifty-first aspects, wherein a finishing stage reactant mixture comprising oxygen is introduced to a finishing stage reactor.

A fifty-third aspect, which is the method of the fifty-second aspects, wherein an oxygen conversion in the finishing stage reactor is equal to or greater than about 99%.

A fifty-fourth aspect, which is the method of any one of the forty-seventh through the fifty-third aspects, wherein a selectivity to C₂₊ hydrocarbons is increased by equal to or greater than about 5% when compared to a selectivity to C₂₊ hydrocarbons of an otherwise similar oxidative coupling of methane reaction conducted in a single stage reactor.

A fifty-fifth aspect, which is the method of any one of the forty-seventh through the fifty-fourth aspects, wherein a synthesis gas H₂/CO molar ratio is increased by equal to or greater than about 25%, when compared to a synthesis gas H₂/CO molar ratio produced by an otherwise similar oxidative coupling of methane reaction conducted in a single stage reactor.

A fifty-sixth aspect, which is the method of any one of the first through the fifty-fifth aspects, wherein at least a portion of the recovered synthesis gas mixture is further converted to methane via a methanation process.

A fifty-seventh aspect, which is the method of any one of the first through the fifty-sixth aspects, wherein at least a portion of the unreacted methane is recovered and recycled to the reactant mixture.

A fifty-eighth aspect, which is a method for producing ethylene and synthesis gas comprising (a) introducing a reactant mixture to a reactor, wherein the reactant mixture comprises methane (CH₄), oxygen (O₂) and hydrogen (H₂), wherein the reactant mixture is characterized by a CH₄/O₂ molar ratio of from about 14:1 to about 18:1, wherein the reactant mixture is characterized by a CH₄/H₂ molar ratio of from about 8:1 to about 15:1, wherein the reactant mixture is characterized by a O₂/H₂ molar ratio of from about 5:1 to about 8:1, wherein the reactor is characterized by a reaction temperature of from about 950° C. to about 1,000° C., wherein the reactor is characterized by a residence time of from about 250 milliseconds to about 750 milliseconds, and wherein the reactor excludes a catalyst; (b) allowing at least a portion of the reactant mixture to react via an oxidative coupling of CH₄ reaction to form a product mixture, wherein the product mixture comprises primary products and unreacted methane, wherein the primary products comprise C₂₊ hydrocarbons and synthesis gas, wherein the C₂₊ hydrocarbons comprise ethylene, and wherein a selectivity to primary products is from about 90% to about 99%; (c) recovering at least a portion of the product mixture from the reactor; and (d) separating at least a portion of the ethylene from the product mixture by cryogenic distillation to yield recovered ethylene.

A fifty-ninth aspect, which is the method of the fifty-eighth aspect, wherein the synthesis gas is separated from the product mixture by cryogenic distillation to yield recovered synthesis gas.

A sixtieth aspect, which is a method for producing olefins and synthesis gas comprising (a) introducing a reactant mixture to a reactor, wherein the reactant mixture comprises methane (CH₄), oxygen (O₂), and hydrogen (H₂) used as an initiator of gas phase reactions, wherein the reactor is characterized by a reaction temperature of from about 700° C. to about 1,100° C.; (b) allowing at least a portion of the reactant mixture to react via an oxidative coupling of CH₄ reaction to form a product mixture, wherein the product mixture comprises primary products and unreacted methane, wherein the primary products comprise C₂₊ hydrocarbons and synthesis gas, wherein the C₂₊ hydrocarbons comprise olefins, and wherein a selectivity to primary products is from about 70% to about 99%; and (c) recovering at least a portion of the product mixture from the reactor.

While aspects of the disclosure have been shown and described, modifications thereof can be made without departing from the spirit and teachings of the invention. The aspects and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Each and every claim is incorporated into the specification as an aspect of the present invention. Thus, the claims are a further description and are an addition to the detailed description of the present invention. The disclosures of all patents, patent applications, and publications cited herein are hereby incorporated by reference.

What is claimed is:

1. A method for producing olefins and synthesis gas comprising:
(a) introducing a reactant mixture to a reactor, wherein the reactant mixture comprises methane (CH₄) and oxygen (O₂), wherein the reactor is characterized by a reaction temperature of from about 700° C. to about 1,100° C.;
(b) allowing at least a portion of the reactant mixture to react via an oxidative coupling of CH₄ reaction to form a product mixture, wherein the product mixture comprises primary products and unreacted methane, wherein the primary products comprise C₂₊ hydrocarbons and synthesis gas, wherein the C₂₊ hydrocarbons comprise olefins, and wherein a selectivity to primary products is from about 70% to about 99%; and
(c) recovering at least a portion of the product mixture from the reactor.

2. The method of claim 1, wherein the reactor is characterized by a residence time of from about 100 milliseconds to about 30 seconds.

3. The method of claim 1, wherein the reactant mixture is characterized by a CH₄/O₂ molar ratio of from about 2:1 to about 40:1.

4. The method of claim 1, wherein the reactant mixture further comprises hydrogen (H₂).

5. The method of claim 4, wherein the reactant mixture is characterized by a CH₄/H₂ molar ratio of from about 10:1 to about 100:1.

6. The method of claim 5, wherein the reactant mixture is characterized by an O₂/H₂ molar ratio of from about 2:1 to about 10:1.

7. The method of claim 6, wherein the reactant mixture is characterized by a (CH₄+H₂)/O₂ molar ratio of from about 2:1 to about 40:1.

8. The method of claim 1, wherein the reactor excludes a catalyst.

9. The method of claim 1, wherein a methane conversion is from about 5% to about 25%.

10. The method of claim 1, wherein a selectivity to C₂₊ hydrocarbons is from about 15% to about 75%.

11. The method of claim 1, wherein a selectivity to ethylene is from about 10% to about 60%.

12. The method of claim 1, wherein equal to or greater than about 10 mol % of the reactant mixture is converted to synthesis gas.

13. The method of claim 1, wherein the synthesis gas is characterized by a hydrogen (H₂) to carbon monoxide (CO) molar ratio of from about 0.5:1 to about 2:1.

14. The method of claim 1, wherein a selectivity to CO is from about 25% to about 85%.

15. The method of claim 1, wherein the reactor comprises from about 2 to about 10 reactors in series, wherein an initial stage reactant mixture comprising methane, oxygen and optionally hydrogen is introduced to an initial stage reactor, and wherein an oxygen conversion in the initial stage reactor is from equal to or greater than about 50% to equal to or less than about 99%.

16. The method of claim 15, wherein an intermediate stage reactant mixture comprising oxygen and optionally hydrogen is introduced to an intermediate stage reactor and wherein an oxygen conversion in the intermediate stage reactor is from equal to or greater than about 50% to equal to or less than about 99%.

17. The method of claim 16, wherein a finishing stage reactant mixture comprising oxygen is introduced to a finishing stage reactor and wherein an oxygen conversion in the finishing stage reactor is equal to or greater than about 99%.

18. A method for producing ethylene and synthesis gas comprising:
(a) introducing a reactant mixture to a reactor, wherein the reactant mixture comprises methane (CH₄), oxygen (O₂) and hydrogen (H₂), wherein the reactant mixture is characterized by a CH₄/O₂ molar ratio of from about 14:1 to about 18:1, wherein the reactant mixture is characterized by a CH₄/H₂ molar ratio of from about 8:1 to about 15:1, wherein the reactant mixture is characterized by a 0₂/H₂ molar ratio of from about 5:1 to about 8:1, wherein the reactor is characterized by a reaction temperature of from about 950° C. to about 1,000° C., wherein the reactor is characterized by a residence time of from about 250 milliseconds to about 750 milliseconds, and wherein the reactor excludes a catalyst;
(b) allowing at least a portion of the reactant mixture to react via an oxidative coupling of CH₄ reaction to form a product mixture, wherein the product mixture comprises primary products and unreacted methane, wherein the primary products comprise C₂₊ hydrocarbons and synthesis gas, wherein the C₂₊ hydrocarbons comprise ethylene, and wherein a selectivity to primary products is from about 90% to about 99%;
(c) recovering at least a portion of the product mixture from the reactor; and
(d) separating at least a portion of the ethylene from the product mixture by cryogenic distillation to yield recovered ethylene.

19. The method of claim 18, wherein the synthesis gas is separated from the product mixture by cryogenic distillation to yield recovered synthesis gas.

20. A method for producing olefins and synthesis gas comprising:
(a) introducing a reactant mixture to a reactor, wherein the reactant mixture comprises methane (CH₄), oxygen (O₂), and hydrogen (H₂) used as an initiator of gas phase reactions, wherein the reactor is characterized by a reaction temperature of from about 700° C. to about 1,100° C.;
(b) allowing at least a portion of the reactant mixture to react via an oxidative coupling of CH₄ reaction to form a product mixture, wherein the product mixture comprises primary products and unreacted methane, wherein the primary products comprise C₂₊ hydrocarbons and synthesis gas, wherein the C₂₊ hydrocarbons comprise olefins, and wherein a selectivity to primary products is from about 70% to about 99%; and
(c) recovering at least a portion of the product mixture from the reactor.
